(19) United States
(12) Patent Application Publication
(10) Pub. No.: US 2009/0148491 A1
(43) Pub. Date: Jun. 11, 2009

(54) Dual-Targeted Drug Carriers

(75) Inventors: Syed F.A. Hossainy, Fremont, CA (US); Dariush Davalian, San Jose, CA (US); Jinping Wan, Sunnyvale, CA (US); Hong Ma, Mountain View, CA (US)

Correspondence Address: SQUIRE, SANDERS & DEMPSEY LLP, 1 MARITIME PLAZA, SUITE 300, SAN FRANCISCO, CA, 94111, US

(73) Assignee: Abbott Cardiovascular Systems Inc., Santa Clara, CA (US)

(21) Appl. No.: 11/951,152

(22) Filed: Dec. 5, 2007

(51) Int. Cl. A61K 9/00; A61P 9/10; A61P 9/00

(52) U.S. Cl. 424/423

(57) ABSTRACT

The present invention relates to implantable medical devices containing surface-treated, dual-targeted drug carriers for treating vascular diseases.

### FIELD

This invention relates to the fields of organic chemistry, pharmaceutical chemistry, polymer science, material science and medicine. In particular, it relates to a medical device and method using dual-targeted drug carriers for treating vascular diseases.

### BACKGROUND

Until the mid-1980s, the accepted treatment for atherosclerosis, i.e., narrowing of the coronary artery(ies) was coronary by-pass surgery. While being quite effective and having evolved to a relatively high degree of safety for such an invasive procedure, by-pass surgery still involves potentially serious complications and in the best of cases an extended recovery period.

With the advent of percutaneous transluminal coronary angioplasty (PTCA) in 1977, the scene changed dramatically. Using catheter techniques originally developed for heart exploration, inflatable balloons were employed to re-open occluded regions in arteries. The procedure was relatively non-invasive, took a very short time compared to by-pass surgery and the recovery time was minimal. However, PTCA brought with it other problems such as vasospasm and elastic recoil of the stretched arterial wall which could undo much of what was accomplished and, in addition, it created a new problem, restenosis, the re-clogging of the treated artery due to neointimal hyperplasia.

The next improvement, advanced in the mid-1980s, was the use of a stent to maintain the luminal diameter after PTCA. This for all intents and purposes put an end to vasospasm and elastic recoil but did not entirely resolve the issue of restenosis. That is, prior to the introduction of stents, restenosis occurred in from about 30 to 50% of patients undergoing PTCA. Stenting reduced this to about 15 to 20%, much improved but still more than desirable.

In 2003, drug-eluting stents or DESs were introduced. The drugs initially employed with the DES were cytostatic compounds, that is, compounds that curtailed the proliferation of cells that resulted in restenosis. The occurrence of restenosis was thereby reduced to about 5 to 7%, a relatively acceptable figure. However, the use of DESs engendered yet another complication, late stent thrombosis, the forming of blood clots long after the stent was in place. It was hypothesized that the formation of blood clots was most likely due to delayed healing, a side-effect of the use of cytostatic drugs.

It has been found that the physiopathology of restenosis involves early injury to smooth muscle cells (SMCs), de-endothelialization and thrombus deposition. Over time, this leads to SMC proliferation and migration and extra-cellular matrix deposition. There is an increasing body of evidence suggesting that inflammation plays a pivotal role in linking this early vascular injury with neointimal growth and eventual lumen compromise, i.e., restenosis. Further, it has been observed that, when stenting is used, the inflammatory state if often more intense and prolonged thus exacerbating the preceding effects.

What is needed is an implantable medical device and method that deals with surface-treated drug carriers which enhance the transport of the particles into a lipid-rich atherosclerotic lesion and enhance the uptake of the particles into macrophages within the lesion for treating the vascular diseases. The current invention provides such devices and methods.

### SUMMARY

Thus, in one aspect, the current invention relates to an implantable medical device, comprising:
a device body having an exposed surface;
a drug reservoir layer disposed over at least a portion of the exposed surface of the device body;
a plurality of particles embedded in the drug reservoir layer;
one or more therapeutic agents encapsulated in the plurality of particles, wherein the particles are surface-treated with a first substance capable of enhancing transport of the particles into a lipid-rich atherosclerotic lesion and a second substance capable of enhancing uptake of the particles into macrophages within the lesion.

In an aspect of this invention, the implantable medical device is a stent.

In an aspect of this invention, the plurality of particles are selected from the group consisting of micelles, worm micelles, liposomes, polymerosomes, hydrogel particles and polymer particles.

In an aspect of this invention, the liposome has a particle size from about 80 nm to about 1 micron.

In an aspect of this invention, the first substance is selected from the group consisting of thiolated chitosan, TDMAC, PPAA, and combination thereof.

In an aspect of this invention, the second substance is selected from the group consisting of phospholipids, DSPG, PLA/PLGA, ceramide, and combination thereof.

As an aspect of this invention, is a method of treating a vascular disease, comprising:
deploying in the vasculature of a patient in need thereof an implantable medical device, wherein the device comprises:
a device body having an exposed surface;
a drug reservoir layer disposed over at least a portion of the exposed surface of the device body;
a plurality of particles embedded in the drug reservoir layer;
one or more therapeutic agents encapsulated in the plurality of particles, wherein the particles are surface-treated with a first substance capable of enhancing transport of the particles into a lipid-rich atherosclerotic lesion and a second substance capable of enhancing uptake of the particles into macrophages within the lesion.

In an aspect of this invention, the implantable medical device is a stent.

In an aspect of this invention, the plurality of particles are selected from the group consisting of a liposome, a micelle, a polymerosome, hydrogel particles and polymer particles.

In an aspect of this invention, the liposome has a particle size from about 80 nm to about 1 micron.

In an aspect of this invention, the first substance is selected from the group consisting of thiolated chitosan, TDMAC, PPAA, and combination thereof.

In an aspect of this invention, the second substance is selected from the group consisting of phospholipids, DSPG, PLA/PLGA, ceramide, and combination thereof.

In an aspect of this invention, the vascular disease is atherosclerosis.

In an aspect of this invention, the vascular disease is restenosis.

In an aspect of this invention, the vascular disease is vulnerable plaque.

In an aspect of this invention, the vascular disease is peripheral vascular disease.

In an aspect of this invention, the vascular disease is late stent thrombosis.

### BRIEF DESCRIPTION OF THE DRAWINGS

The figures are provided as examples of certain embodiments of this invention to aid in its understanding and are not intended nor are they to be construed as limiting the scope of the invention in any manner whatsoever.

FIG. 1 depicts an uptake of liposomes by macrophage using fluorescein isothiocyante (FITC) as a reference standard at excitation/emission (Ex/Em) 490 nm/520 nm for 4 hours (black bar) and 24 hours (white bar).

FIG. 2 depicts no uptake of liposomes by human cardiovascular arterial smooth muscle cells (HCASMC) using fluorescein isothiocyante (FITC) as a reference standard at excitation/emission (Ex/Em) 490 nm/520 nm for 4 hours (black bar) and 24 hours (white bar).

FIG. 3 depicts no uptake of liposomes by human cardiovascular arterial endothelial cells (HCAEC) using fluorescein isothiocyante (FITC) as a reference standard at excitation/emission (Ex/Em) 490 nm/520 nm for 4 hours (black bar) and 24 hours (white bar).

FIG. 4 depicts an uptake of PLGA nanoparticles by macrophage (J774) using cyanine dye, CY5 as a reference standard at excitation/emission (Ex/Em) 649 nm/666 nm for 4 hours (white bar) and 24 hours (black bar).

FIG. 5 depicts no uptake of PLGA nanoparticles by human cardiovascular arterial smooth muscle cells (HCASMC) using cyanine dye, CY5 as a reference standard at excitation/emission (Ex/Em) 649 nm/666 nm for 4 hours (white bar) and 24 hours (black bar).

### DETAILED DESCRIPTION

Use of the singular herein includes the plural and visa versa unless expressly stated to be otherwise. That is, “a” and “the” refer to one or more of whatever the word modifies. For example, “a therapeutic agent” includes one such agent, two such agents, etc. Likewise, “the layer” may refer to one, two or more layers and “the polymer” may mean one polymer or a plurality of polymers. By the same token, words such as, without limitation, “layers” and “polymers” would refer to one layer or polymer as well as to a plurality of layers or polymers unless, again, it is expressly stated or obvious from the context that such is not intended.

As used herein, a “device” refers to any manner of apparatus that is used or that may be used to in conjunction with a delivery interface of this invention. The device may be transitory, that is, it may be a device that is inserted into a patient's body for only so long as is necessary to administer a therapeutic agent to the patient from a delivery interface of the device or it may be an implantable medical device intended to remain in a patient's body for longer than necessary to deliver the therapeutic agent, possibly for as long as the remaining lifetime of the patient. Intermediate between transitory devices and implantable medical devices intended to remain in place permanently are biodegradable implantable medical devices which over time degrade to substances that can either be adsorbed into or excreted by the body.

An example, without limitation, of a transitory device is a vascular catheter. A vascular catheter is a thin, flexible tube with a manipulating means at one end, referred to as the proximal end, which remains outside the patient's body, and an operative device at or near the other end, called the distal end, which is inserted into the patient's artery or vein. The catheter is often introduced into a patient's vasculature at a point remote from the target site, e.g., into the femoral artery of the leg where the target is in the vicinity of the heart. The catheter is steered, assisted by a guide wire than extends through a lumen in the flexible tube, to the target site whereupon the guide wire is withdrawn at which time the lumen may be used for the introduction of fluids, often containing therapeutic agents, to the target site.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves and cerebrospinal fluid shunts. An implantable medical device specifically designed and intended solely for the localized delivery of a therapeutic agent is within the scope of this invention.

As used herein, “device body” refers to a fully formed implantable medical with an outer surface to which no coating or layer of material different from that of which the device itself is manufactured has been applied. By “exposed surface” of a device body is meant any surface however spatially oriented that is in contact with bodily tissue or fluids. A common example of a “device body” is a BMS, i.e., a bare metal stent, which, as the name implies, is a fully-formed usable stent that has not been coated with a layer of any material different from the metal of which it is made on any surface that is in contact with bodily tissue or fluids. Of course, device body refers not only to BMSs but to any uncoated device regardless of what it is made of.

Implantable medical devices made of virtually any material, i.e., materials presently known to be useful for the manufacture of implantable medical devices and materials that may be found to be so in the future, may be used with a coating of this invention. For example, without limitation, an implantable medical device useful with this invention may be made of one or more biocompatible metals or alloys thereof including, but not limited to, cobalt-chromium alloy (ELGILOY, L-605), cobalt-nickel alloy (MP-35N), 316L stainless steel, high nitrogen stainless steel, e.g., BIODUR 108, nickel-titanium alloy (NITINOL), tantalum, platinum, platinum-iridium alloy, gold and combinations thereof.

Implantable medical devices may also be made of polymers that are biocompatible and biostable or biodegradable, the latter term including bioabsorbable and/or bioerodable.

As used herein, “biocompatible” refers to a polymer that both in its intact, as synthesized state and in its decomposed state, i.e., its degradation products, is not, or at least is minimally, toxic to living tissue; does not, or at least minimally and reparably, injure(s) living tissue; and/or does not, or at least minimally and/or controllably, cause(s) an immunological reaction in living tissue.

Among useful biocompatible, relatively biostable polymers are, without limitation, polyacrylates, polymethacryates, polyureas, polyurethanes, polyolefins, polyvinylhalides, polyvinylidenehalides, polyvinylethers, polyvinylaromatics, polyvinylesters, polyacrylonitriles, alkyd resins, polysiloxanes and epoxy resins.

Biocompatible, biodegradable polymers include naturally-occurring polymers such as, without limitation, collagen, chitosan, alginate, fibrin, fibrinogen, cellulosics, starches, dextran, dextrin, hyaluronic acid, heparin, glycosaminoglycans, polysaccharides and elastin.

One or more synthetic or semi-synthetic biocompatible, biodegradable polymers may also be used to fabricate an implantable medical device useful with this invention. As used herein, a synthetic polymer refers to one that is created wholly in the laboratory while a semi-synthetic polymer refers to a naturally-occurring polymer than has been chemically modified in the laboratory. Examples of synthetic polymers include, without limitation, polyphosphazines, polyphosphoesters, polyphosphoester urethane, polyhydroxyacids, polyhydroxyalkanoates, polyanhydrides, polyesters, polyorthoesters, polyamino acids, polyoxymethylenes, poly(ester-amides) and polyimides.

Blends and copolymers of the above polymers may also be used and are within the scope of this invention. Based on the disclosures herein, those skilled in the art will recognize those implantable medical devices and those materials from which they may be fabricated that will be useful with the coatings of this invention.

At present, preferred implantable medical devices for use with the coatings of this invention are stents.

As used herein, a “stent” refers generally to any device used to hold tissue in place in a patient's body. Particularly useful stents, however, are those used for the maintenance of the patency of a vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease such as atherosclerosis, restenosis and vulnerable plaque. Vulnerable plaque (VP) refers to a fatty build-up in an arterial wall thought to be caused by inflammation. The VP is covered by a thin fibrous cap that can rupture leading to blood clot formation. A stent can be used to strengthen the wall of the vessel in the vicinity of the VP and act as a shield against such rupture. A stent can be used in, without limitation, neuro, carotid, coronary, pulmonary, aorta, renal, biliary, iliac, femoral and popliteal as well as other peripheral vasculatures. A stent can be used in the treatment or prevention of disorders such as, without limitation, thrombosis, restenosis, hemorrhage, vascular dissection or perforation, vascular aneurysm, chronic total occlusion, claudication, anastomotic proliferation, bile duct obstruction and ureter obstruction.

In addition to the above uses, stents may also be employed for the localized delivery of therapeutic agents to specific treatment sites in a patient's body. In fact, therapeutic agent delivery may be the sole purpose of the stent or the stent may be primarily intended for another use such as those discussed above with drug delivery providing an ancillary benefit.

A stent used for patency maintenance is usually delivered to the target site in a compressed state and then expanded to fit the vessel into which it has been inserted. Once at a target location, a stent may be self-expandable or balloon expandable. In any event, due to the expansion of the stent, any coating thereon must be flexible and capable of elongation.

As used herein, “therapeutic agent” refers to any substance that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to retrogress; or, (4) alleviating one or more symptoms of the disease. As used herein, a therapeutic agent also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a retrogressed level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded.

As used herein, the terms “drug” and “therapeutic agent” are used interchangeably.

As used herein, “treating” refers to the administration of a therapeutically effective amount of a therapeutic agent to a patient known or suspected to be suffering from a vascular disease.

A “therapeutically effective amount” refers to that amount of a therapeutic agent that will have a beneficial affect, which may be curative or palliative, on the health and well-being of the patient with regard to the vascular disease with which the patient is known or suspected to be afflicted. A therapeutically effective amount may be administered as a single bolus, as intermittent bolus charges, as short, medium or long term sustained release formulations or as any combination of these. As used herein, short-term sustained release refers to the administration of a therapeutically effective amount of a therapeutic agent over a period from about several hours to about 3 days. Medium-term sustained release refers to administration of a therapeutically effective amount of a therapeutic agent over a period from about 3 day to about 14 days and long-term refers to the delivery of a therapeutically effective amount over any period in excess of about 14 days.

As used herein, a “vascular disease” refers to a disease of the vessels, primarily arteries and veins, which transport blood to and from the heart, brain and peripheral organs such as, without limitation, the arms, legs, kidneys and liver. In particular “vascular disease” refers to the coronary arterial system, the carotid arterial system and the peripheral arterial system. The disease that may be treated is any that is amenable to treatment with a therapeutic agent, either as the sole treatment protocol or as an adjunct to other procedures such as surgical intervention. The disease may be, without limitation, atherosclerosis, vulnerable plaque, restenosis or peripheral arterial disease. “Atherosclerosis” refers to the depositing of fatty substances, cholesterol, cellular waste products, calcium and fibrin on the inner lining or intima of an artery. Smooth muscle cell proliferation and lipid accumulation accompany the deposition process. In addition, inflammatory substances that tend to migrate to atherosclerotic regions of an artery are thought to exacerbate the condition. The result of the accumulation of substances on the intima is the formation of fibrous (atheromatous) plaques that occlude the lumen of the artery, a process called stenosis. When the stenosis becomes severe enough, the blood supply to the organ supplied by the particular artery is depleted resulting is strokes, if the afflicted artery is a carotid artery, heart attack if the artery is a coronary artery, or loss of organ function if the artery is peripheral. “Restenosis” refers to the re-narrowing or blockage of an artery at or near the site where angioplasty or another surgical procedure was previously performed to remove a stenosis. It is generally due to smooth muscle cell proliferation and, at times, is accompanied by thrombosis. Prior to the advent of implantable stents to maintain the patency of vessels opened by angioplasty, restenosis occurred in 40-50% of patients within 3 to 6 months of undergoing the procedure. Post-angioplasty restenosis before stents was due primarily to smooth muscle cell proliferation. There were also issues of acute reclosure due to vasospasm, dissection, and thrombosis at the site of the procedure. Stents eliminated acute closure from vasospasm and greatly reduced complications from dissections. While the use of IIb-IIIa anti-platelet drugs such as abciximab and epifabatide, which are anti-thrombotic, reduced the occurrence of post-procedure clotting (although stent placement itself can initiate thrombosis). Stent placement sites are also susceptible to restenosis due to abnormal tissue growth at the site of implantation. This form of restenosis tends also to occur at 3 to 6 months after stent placement but it is not affected by the use of anti-clotting drugs. Thus, alternative therapies are continuously being sought to mitigate, preferably eliminate, this type of restenosis. Drug eluting stents (DES) which release a variety of therapeutic agents at the site of stent placement have been in use for some time. To date these stents comprised delivery interfaces (lengths) that are less than 40 mm in length and, in any event, have delivery interfaces that are not intended, and most often do not, contact the luminal surface of the vessel at the non-afflicted region at the periphery of the afflicted region.

“Vulnerable plaque” refers to an atheromatous plaque that has the potential of causing a thrombotic event and is usually characterized by a very thin wall separating it from the lumen of an artery. The thinness of the wall renders the plaque susceptible to rupture. When the plaque ruptures, the inner core of usually lipid-rich plaque is exposed to blood, with the potential of causing a potentially fatal thrombotic event through adhesion and activation of platelets and plasma proteins to components of the exposed plaque.

The phenomenon of “vulnerable plaque” has created new challenges in recent years for the treatment of heart disease. Unlike occlusive plaques that impede blood flow, vulnerable plaque develops within the arterial walls, but it often does so without the characteristic substantial narrowing of the arterial lumen which produces symptoms. As such, conventional methods for detecting heart disease, such as an angiogram, may not detect vulnerable plaque growth into the arterial wall.

The intrinsic histological features that may characterize a vulnerable plaque include increased lipid content, increased macrophage, foam cell and T lymphocyte content, and reduced collagen and smooth muscle cell (SMC) content. This fibroatheroma type of vulnerable plaque is often referred to as “soft,” having a large lipid pool of lipoproteins surrounded by a fibrous cap. The fibrous cap contains mostly collagen, whose reduced concentration combined with macrophage-derived enzyme degradation can cause the fibrous cap of these lesions to rupture under unpredictable circumstances. When ruptured, the lipid core contents, thought to include tissue factor, contact the arterial bloodstream, causing a blood clot to form that can completely block the artery resulting in an acute coronary syndrome (ACS) event. This type of atherosclerosis is coined “vulnerable” because of unpredictable tendency of the plaque to rupture. It is thought that hemodynamic and cardiac forces, which yield circumferential stress, shear stress, and flexion stress, may cause disruption of a fibroatheroma type of vulnerable plaque. These forces may rise as the result of simple movements, such as getting out of bed in the morning, in addition to in vivo forces related to blood flow and the beating of the heart. It is thought that plaque vulnerability in fibroatheroma types is determined primarily by factors which include: (1) size and consistency of the lipid core; (2) thickness of the fibrous cap covering the lipid core; and (3) inflammation and repair within the fibrous cap.

“Thrombosis” refers to the formation or presence of a blood clot (thrombus) inside a blood vessel or chamber of the heart. A blood clot that breaks off and travels to another part of the body is called an embolus. If a clot blocks a blood vessel that feeds the heart, it causes a heart attack. If a clot blocks a blood vessel that feeds to brain, it causes a stroke. “Late stent thrombosis” refers to the formation of a blood clot (thrombus) which occurs usually after 30 days after stent implantation. Late stent thrombosis may occur months or even years after stent implantation.

Peripheral vascular diseases are generally caused by structural changes in blood vessels caused by such conditions as inflammation and tissue damage. A subset of peripheral vascular disease is peripheral artery disease (PAD). PAD is a condition that is similar to carotid and coronary artery disease in that it is caused by the buildup of fatty deposits on the lining or intima of the artery walls. Just as blockage of the carotid artery restricts blood flow to the brain and blockage of the coronary artery restricts blood flow to the heart, blockage of the peripheral arteries can lead to restricted blood flow to the kidneys, stomach, arms, legs and feet.

Suitable therapeutic agents include, without limitation, antiproliferative agents, anti-inflammatory agents, antineoplastics and/or antimitotics, antiplatelet, anticoagulant, antifibrin, and antithrombin drugs, cytostatic or antiproliferative agents, antibiotics, antiallergic agents, antioxidants and other bioactive agents known to those skilled in the art.

Examples of antiproliferative agents include, without limitation, actinomycins, taxol, docetaxel, paclitaxel, rapamycin, 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin, everolimus, biolimus, perfenidone and derivatives, analogs, prodrugs, co-drugs and combinations of any of the foregoing.

Examples of anti-inflammatory agents include both steroidal and non-steroidal (NSAID) anti-inflammatory agents such as, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecrolimus and derivatives, analogs, prodrugs, co-drugs and combinations of any of the foregoing.

Examples of antineoplastics and antimitotics include, without limitation, paclitaxel, docetaxel, methotrexate, azathioprine, vincristine, vinblastine, fluorouracil, doxorubicin hydrochloride, and mitomycin.

Examples of antiplatelet, anticoagulant, antifibrin, and antithrombin drugs include, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as Angiomax ä, calcium channel blockers such as nifedipine, colchicine, fish oil (omega 3-fatty acid), histamine antagonists, lovastatin, monoclonal antibodies such as those specific for Platelet-Derived Growth Factor (PDGF) receptors, nitroprusside, phosphodiesterase inhibitors, prostaglandin inhibitors, suramin, serotonin blockers, steroids, thioprotease inhibitors, triazolopyrimidine, nitric oxide or nitric oxide donors, super oxide dismutases, super oxide dismutase mimetic, 4-amino-2,2,6,6-tetramethylpiperidine-1-oxyl (4-amino-TEMPO) and derivatives, analogs, prodrugs, codrugs and combinations thereof.

Examples of cytostatic or antiproliferative agents include, without limitation, angiopeptin, angiotensin converting enzyme inhibitors such as captopril, cilazapril or lisinopril, calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide.

Examples of antiallergic agents include, without limitation, permirolast potassium.

Other compounds that may be used as bioactive agents of this invention include, without limitation, alpha-interferon, genetically engineered epithelial cells, dexamethasone, antisense molecules which bind to complementary DNA to inhibit transcription, and ribozymes, antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy; antiviral agents; analgesics and analgesic combinations; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and derivatives, analogs, prodrugs, codrugs and combinations of any of the foregoing.

Other bioactive agents include a corticosteroid, everolimus, zotarolimus, sirolimus, and derivatives thereof, paclitaxel, biolimus A9, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase.

As used herein, a “primer layer” refers to a coating consisting of a polymer or blend of polymers that exhibit good adhesion characteristics with regard to the material of which the device body is manufactured and good adhesion characteristic with regard to whatever material is to be coated on the device body. Thus, a primer layer serves as an intermediary layer between a device body and materials to be affixed to the device body and is, therefore, applied directly to the device body. Examples without limitation, of primers include acrylate and methacrylate polymers with poly(n-butyl methacrylate) being a presently preferred primer. Some additional examples of primers include, but are not limited to, poly(ethylene-co-vinyl alcohol), poly(vinyl acetate-co-vinyl alcohol), poly(methacrylates), poly(acrylates), polyethyleneamine, polyallylamine, chitosan, poly(ethylene-co-vinyl acetate), and parylene-C.

As use herein, a material that is described as a layer “disposed over” an indicated substrate, e.g., without limitation, a device body or another layer, refers to a relatively thin coating of the material applied, preferably at present, directly to essentially the entire exposed surface of the indicated substrate. By “exposed surface” is meant that surface of the substrate that, in use, would be in contact with bodily tissues or fluids. “Disposed over” may, however, also refer to the application of the thin layer of material to an intervening layer that has been applied to the substrate, wherein the material is applied in such a manner that, were the intervening layer not present, the material would cover substantially the entire exposed surface of the substrate.

As used herein, “drug reservoir layer” refers either to a layer of one or more therapeutic agents applied neat or to a layer of polymer or blend of polymers that has dispersed within its three-dimensional structure one or more therapeutic agents. A polymeric drug reservoir layer is designed such that, by one mechanism or another, e.g., without limitation, by elution or as the result of biodegradation of the polymer, the therapeutic substance is released from the layer into the surrounding environment. For the purpose of this invention, the drug reservoir layer also acts as rate-controlling layer. As used herein, “rate-controlling layer” refers to a polymer layer that controls the release of therapeutic agents or drugs into the environment. The drug reservoir of this invention comprises a plurality of particles (drug carriers) embedded in the drug reservoir layer. The therapeutic agents are encapsulated within the particles. Presently preferred particles of this invention include, but are not limited to, micelles, liposomes, polymerosomes, hydrogel particles and polymer particles.

As used herein, a “micelle” refers a spherical colloidal nanoparticle spontaneous formed by many amphiphilic molecules in an aqueous medium when the Critical Micelle Concentration (CMC) is exceeded. Amphiphilic molecules have two distinct components, differing in their affinity for a solute, most particularly water. The part of the molecule that has an affinity for water, a polar solute, is said to be hydrophilic. The part of the molecule that has an affinity for non-polar solutes such as hydrocarbons is said to be hydrophobic. When amphiphilic molecules are placed in water, the hydrophilic moiety seeks to interact with the water while the hydrophobic moiety seeks to avoid the water. To accomplish this, the hydrophilic moiety remains in the water while the hydrophobic moiety is held above the surface of the water in the air or in a non-polar, non-miscible liquid floating on the water. The presence of this layer of molecules at the water's surface disrupts the cohesive energy at the surface and lowers surface tension. Amphiphilic molecules that have this effect are known as “surfactants.” Only so many surfactant molecules can align as just described at the water/air or water/hydrocarbon interface. When the interface becomes so crowded with surfactant molecules that no more can fit in, i.e., when the CMC is reached, any remaining surfactant molecules will form into spheres with the hydrophilic ends of the molecules facing out, that is, in contact with the water forming the micelle corona and with the hydrophobic “tails” facing toward the center of the of the sphere. Therapeutic agents suspended in the aqueous medium can be trapped within the chamber formed by the surfactant molecules. Because of their nanoscale size, generally from about 5 nm to about 50 nm, micelles have been shown to exhibit spontaneous accumulation in pathological areas with leaky vasculature and impaired lymphatic drainage, a phenomenon known as the Enhanced Permeability and Retention or EPR effect.

The problem with micelles formed from relatively low molecular weight surfactants is that their CMC is usually quite high so that the formed micelles dissociate rather rapidly upon dilution, i.e., the molecules head for open places at the surface of the water with the resulting precipitation of the therapeutic agent. Fortunately, this short-coming can be avoided by using lipids with a long fatty acid chain or two fatty acid chains, specifically phospholipids and sphingolipids, or polymers, specifically block copolymers to form the micelles.

Polymeric micelles have been prepared that exhibit CMCs as low as 10⁻⁶ M (molar). Thus, they tend to be very stable while at the same time showing the same beneficial characteristics as surfactant micelles. Any micelle-forming polymer presently known in the art or as such may become known in the future may be used in the method of this invention. Examples of micelle-forming polymers are, without limitation, methoxy poly(ethylene glycol)-b-poly(ε-caprolactone), conjugates of poly(ethylene glycol) with phosphatidyl-ethanolamine, poly(ethylene glycol)-b-polyesters, poly(ethylene glycol)-b-poly(L-aminoacids), poly(N-vinylpyrrolidone)-bl-poly(orthoesters), poly(N-vinylpyrrolidone)-b-polyanhydrides and poly(N-vinylpyrrolidone)-b-poly(alkyl acrylates).

In addition to the classical spherical micelles described above, a particle of this invention may comprise a construct known as a worm micelle. Worm micelles are, as the name suggests, cylindrical in shape rather than spherical. They are prepared by varying the weight fraction of the hydrophilic polymer block to the total block copolymer molecular weight in the hydrophilic polymer-b-hydrophobic polymer structure discussed above for preparing spherical micelles. Worm micelles have the potential advantage of not only being as bio-inert and as stable as spherical polymeric micelles but also of being flexible. Polyethylene oxide has been used extensively to create worm micelles with a number of hydrophobic polymers such as, without limitation, poly(lactic acid), poly(ε-caprolactone), poly(ethylethylene) and polybutadiene. A representative description of worm micelle formation, characterization and drug loading can be found in Kim, Y., et al., Nanotechnology, 2005, 16:S484-S491. The techniques described there as well as any other that is currently known or may become known in the future may be used to create worm micelles useful as vesicles of this invention.

In addition to substantially spherical micelles and worm micelles, a particle of this invention may be a liposome. As used herein, a “liposome” refers to a vesicle consisting of an aqueous core enclosed by one or more phospholipid layers.

Phospholipids are molecules that have two primary regions, a hydrophilic head region comprised of a phosphate of an organic molecule and one or more hydrophobic fatty acid tails. In particular, naturally-occurring phospholipids have a hydrophilic region comprised of choline, glycerol and a phosphate and two hydrophobic regions comprised of fatty acid. When phospholipids are placed in an aqueous environment, the hydrophilic heads come together in a linear configuration with their hydrophobic tails aligned essentially parallel to one another. A second line of molecules then aligns tail-to-tail with the first line as the hydrophobic tails attempt to avoid the aqueous environment. To achieve maximum avoidance of contact with the aqueous environment, i.e., at the edges of the bilayers, while at the same time minimizing the surface area to volume ratio and thereby achieve a minimal energy conformation, the two lines of phospholipids, know as a phospholipid bilayer or a lamella, converge into a sphere and in doing so entrap some of the aqueous medium, and whatever may be dissolved or suspended in it, in the core of the sphere. Examples of phospholipids that may be used to create liposomes are, without limitation, 1,2-dimyristroyl-sn-glycero-3-phosphocholine, 1,2-dilauroyl-sn-glycero-3-phosphocholine, 1,2-distearoyl-sn-glycero-3-phosphocholine, 1,2-dimyristoyl-sn-glycero-3-phosphoethanolamine, 1,2-dipalmitoyl-sn-glycero-3-phosphoethanolamine, 1,2-dioleoyl-sn-glycero-3-phosphate monosodium salt, 1,2-dipalmitoyl-sn-glycero-3-[phosphor-rac-(1-glycerol)] sodium salt, 1,2-dimyristoyl-sn-glycero-3-[phospho-L-serine] sodium salt, 1,2-dioleoyl-sn-glycero-3-phosphoethanolamine-N-glutaryl sodium salt and 1,1′,2,2′-tetramyristoyl cardiolipin ammonium salt.

Liposomes may be unilamellar, composed of a single bilayer, or they may be multilamellar, composed of two or more concentric bilayers. Liposomes range from about 20-100 nm diameter for small unilamellar vesicles (SUVs), about 100-5000 nm for large multilamellar vesicles and ultimately to about 100 microns for giant multilamellar vesicles (GMVs). LMVs form spontaneously upon hydration with agitation of dry lipid films/cakes which are generally formed by dissolving a lipid in an organic solvent, coating a vessel wall with the solution and evaporating the solvent. Energy is then applied to convert the LMVs to SUVs, LUVs, etc. The energy can be in the form of, without limitation, sonication, high pressure, elevated temperatures and extrusion to provide smaller single and multi-lamellar vesicles. During this process some of the aqueous medium is entrapped in the vesicle. Generally, however, the fraction of total solute and therefore the amount of therapeutic agent entrapped tends to be rather low, typically in the range of a few percent. Recently, liposome preparation by emulsion templating (Pautot, et al., Langmuir, 2003, 19:2870) has been described. Emulsion templating comprises, in brief, the preparation of a water-in-oil emulsion stabilized by a lipid, layering of the emulsion onto an aqueous phase, centrifugation of the water/oil droplets into the water phase and removal of the oil phase to give a dispersion of unilamellar liposomes. This method can be used to make asymmetric liposomes in which the inner and outer monolayers of the single bilayer contain different lipids. Liposomes prepared by any method, not merely those described above, may be used in the compositions and methods of this invention. Any of the preceding techniques as well as any others known in the art or as may become known in the future may be used as compositions of therapeutic agents in or on a delivery interface of this invention. Liposomes comprising phospho- and/or sphingolipids may be used to deliver hydrophilic (water-soluble) or precipitated therapeutic compounds encapsulated within the inner liposomal volume and/or to deliver hydrophobic therapeutic agents dispersed within the hydrophobic bilayer membrane. The presently preferred particle size of liposomes range is from about 80 nm to about 1 micron.

Polymerosomes are liposome-like particles made of natural polymers other than phospholipids or sphingolipids, semi-synthetic polymers, which can include synthetically modified phospholipids and sphingolipids and totally synthetic polymers. Polymerosomes can be prepared in the same manner as liposomes. That is, a film of a diblock copolymer can be formed by dissolving the copolymer in an organic solvent, applying a film of the copolymer-containing solvent to a vessel surface, removing the solvent to leave a film of the copolymer and then hydrating the film. This procedure, however, tends to result is a polydispersion of micelles, worm micelles and vesicles of varying sizes. Polymerosomes can also be prepared by dissolving the diblock copolymer in a solvent and then adding a poor solvent for one of the blocks, which will result in the spontaneous formation of polymerosomes.

As with liposomes, polymerosomes can be used to encapsulate therapeutic agents by including the therapeutic agent in the water used to rehydrate the copolymer film. Polymerosomes can also be force-loaded by osmotically driving the therapeutic agent into the core of the vesicle. Also as with liposomes, the loading efficiency is generally low. Recently, however, a technique has been reported that provides polymerosomes of relative monodispersivity and high loading efficiency; generation of polymerisomes from double emulsions. Lorenceau, et al., Langmuir, 2005, 21:9183-86. The technique involves the use of microfluidic technology to generate double emulsions consisting of water droplets surrounded by a layer of organic solvent. These droplet-in-a-drop structures are then dispersed in a continuous water phase. The diblock copolymer is dissolved in the organic solvent and self-assembles into proto-polymerosomes on the concentric interfaces of the double emulsion. The actual polymerosomes are formed by completely evaporating the organic solvent from the shell. Using this procedure the size of the polymerosomes can be finely controlled and, in addition, the ability to maintain complete separation of the internal fluids from the external fluid throughout the process allows extremely efficient encapsulation. This technique along with any other technique known in the art or as may become known in the future can be used to prepare a composition of therapeutic agents for use in or on a delivery interface of this invention.

As used herein, a “gel” or “hydrogel” refers to a water-insoluble substance that nevertheless is capable of imbibing a substantial amount of water, the substance swelling in the process.

The particles of this invention are surface-treated with at least two types of substances. The first substance is capable of enhancing the transport of the particles into a lipid-rich environment such as, without limitation, an atherosclerotic lesion. Presently preferred first substances of this invention include, but are not limited to, thiolated chitosan, tridodecylmethylammonium chloride (TDMAC), poly(propyl-acrylic acid (PPAA) and combination thereof. The second substance is capable of enhancing uptake of the particles into macrophages such as those that are known to occur within the atherosclerotic lesions. Presently preferred second substances of this invention include, but are not limited to, phospholipids, disteroylphosphatidylglycerol (DSPG), PLA/PLGA, ceramide, and combination thereof. As used herein, a “phospholipid” refers to class of lipid molecules. A phospholipid is composed of a hydrophilic polar head group and a hydrophobic tail. The polar head group contains one or more phosphate groups. The hydrophobic tail is made up of two fatty acyl chains. When many phospholipid molecules are placed in water, their hydrophilic heads tend to face water and the hydrophobic tails are forced to stick together, forming a bilayer. As used herein, a “ceramide” refers to a family of lipid molecules. A ceramide is composed of sphingosine and a fatty acid. Sphingosine (2-amino-4-octadecene-1,3-diol) is an 18-carbon amino alcohol with an unsaturated hydrocarbon chain, which forms a primary part of sphingolipids, a class of cell membrane lipids that include sphingomyelin, an important phospholipid. A fatty acid is a carboxylic acid often with a long unbranched aliphatic tail (chain), which is either saturated or unsaturated.

Llposomes are a currently preferred type of particle of this invention. Various liposomes with different surface properties were thus synthesized and characterized by in vitro macrophage uptake assay. Table 1 shows five different formulations for preparing liposomes comprising disteroylphosphatidylglycerol (DSPG), disteroylphosphatidylcholine (DSPC), cholesterol (Chol.), and ceramide (Cer.).

TABLE 1

| | | | Liposome | | Zeta |
|---|---|---|---|---|---|
| Formulation | | Formulation | Size | | Potential |
| Number | Formulation | Molar Ratio | (nm) | Polydispersity | (meV) |
| 1 | DSPG:DSPC:Chol.:Cer. | 1:3:1:3 | 187 | 0.05 | −27.163 |
| 2 | DSPG:DSPC:Chol.:Cer. | 1:2:1:3 | 203 | 0.05 | −23.848 |
| 3 | DSPG:DSPC:Chol.:Cer. | 1:1.4:1:3 | 177 | 0.06 | −30.514 |
| 4 | DSPG:DSPC:Chol.:Cer. | 1:1:1:3 | 178 | 0.06 | −38.794 |
| 5 | DSPG:DSPC:Chol.:Cer. | 1:1:1:3 | 100 | 0.07 | −36.668 |

DSPG is negatively charged while DSPC, cholesterol and ceramide are neutral.

“Polydispersity” refers to the range of particle sizes in a particular population and is normally presented as the polydispersivity index, PI which is equal to the weight-averaged molecular weight of a polymer divided by its number-averaged molecular weight. A PI of 1.0 means that the polymer is monodisperse, i.e., all the polymer particles are the same size. The stable dispersion of nanoparticles can be achieved by using sufficient surfactant to prevent the particle aggregation.

“Zeta potential” refers to electrokinetic potential in colloidal systems and is a measure of the stability of a colloid. Specifically, zeta potential is the electric potential in the interface double layer at the location of the slipping plane versus a point in the bulk fluid at a distance from the interface double layer. In other words, zeta potential is the electrical potential difference between the dispersing fluid and the stationary layer of fluid attached to the dispersed colloidal particles. Colloids with a high positive or negative zeta potential are electrically stabilized while the particles of now zeta potential colloids tend to coagulate or flocculate.

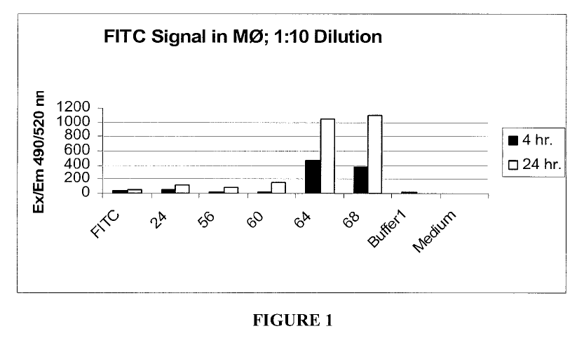

The combination of ceramides with negatively charged surfaces was shown to increase macrophage uptake efficiency but not that of smooth muscle cells or endothelial cells. That is, FIG. 1 shows the uptake of liposomes by macrophage using fluorescein isothiocyante (FITC) as a reference standard. Uptake was measured at 4 hours (black bar) and 24 hours (white bar). The macrophages were cultured on 96 well cell culture plate followed by treating various formulations of liposomes for 4 hours (black bar) or 24 hours (white bar). The macrophages were washed in 1× PBS for three times and then lysed in 50 μl cell lysis buffer for 15 minutes at room temperature. The FITC signal inside the macrophages was evaluated at excitation/emission (Ex/Em) wavelengths of 490 nm/520 nm. The Formulation Number 1 in Table 1 corresponds to 24, Formulation Number 2 corresponds to 56, Formulation Number 3 corresponds to 60, Formulation Number 4 corresponds to 64 and Formulation Number 5 corresponds to 68 in FIG. 1, respectively.

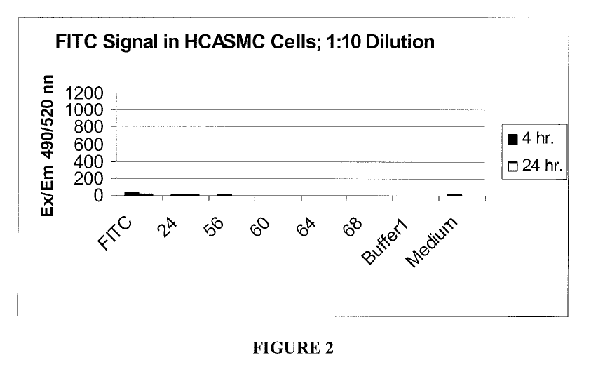

FIG. 2 shows that, under essentially the same conditions, there is no uptake of liposomes by human cardiovascular arterial smooth muscle cells (HCASMC). Once again fluorescein isothiocyante (FITC) was used as the reference standard and results were determined at excitation/emission (Ex/Em) wavelengths of 490 nm/520 nm. The HCASMC were cultured on 96 well cell culture plate followed by treatment with the various formulations of liposomes for 4 hours (black bar) or 24 hours (white bar). The HCASMC were washed in 1× PBS for three times and then lysed in 50 μl cell lysis buffer for 15 minutes at room temperature. The FITC signal inside the HCASMC was evaluated at the indicated excitation/emission wavelengths. The Formulation Number 1 in Table 1 corresponds to 24, Formulation Number 2 corresponds to 56, Formulation Number 3 corresponds to 60, Formulation Number 4 corresponds to 64 and Formulation Number 5 corresponds to 68 in FIG. 2, respectively.

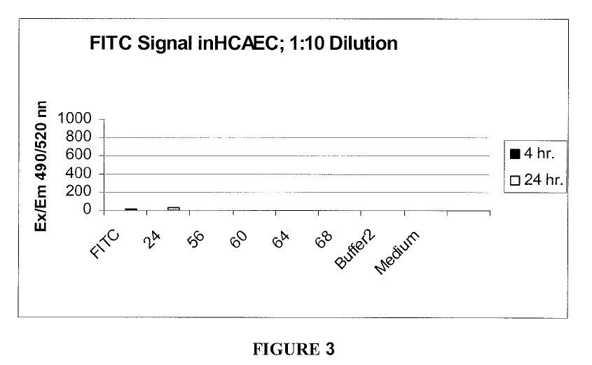

FIG. 3 shows that there likewise was no uptake of liposomes by human cardiovascular arterial endothelial cells (HCAEC). Once again fluorescein isothiocyanate (FITC) was used as reference the standard. The human HCAEC were cultured on 96 well cell culture plate followed by treating various formulations of liposomes for 4 hours (black bar) or 24 hours (white bar). The HCAEC were washed in 1× PBS for three times and then lysed in 50 μl cell lysis buffer for 15 minutes at room temperature. The FITC signal of the contents of the HCAEC was evaluated at excitation/emission (Ex/Em) 490 nm/520 nm. The Formulation Number 1 in Table 1 corresponds to 24, Formulation Number 2 corresponds to 56, Formulation Number 3 corresponds to 60, Formulation Number 4 corresponds to 64 and Formulation Number 5 corresponds to 68 in FIG. 3, respectively.

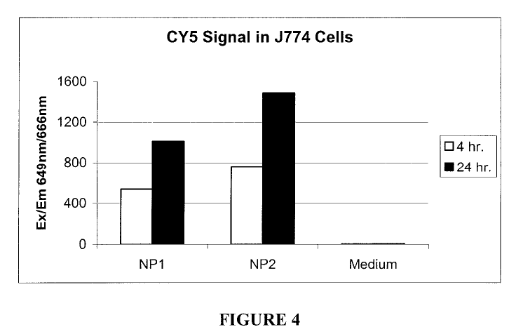

FIG. 4 shows the uptake of PLGA nanoparticles by macrophage (J774) using the cyanine dye, CY5 (manufactured by GE Healthcare) as a reference standard. The macrophages were cultured on 96 well cell culture plate followed by treatment with various formulations of PLGA nanoparticles for 4 hours (white bar) or 24 hours (black bar). The macrophages were washed in 1× PBS three times and then lysed in 50 μl cell lysis buffer for 15 minutes at room temperature. The CY5 signal of the macrophage contents was evaluated at excitation/emission (Ex/Em) 649 nm/666 nm. The NP1 refers to nanoparticles having particle size 600 nm and NP2 refers to nanoparticles having particle size 1100 nm in FIG. 4, respectively.

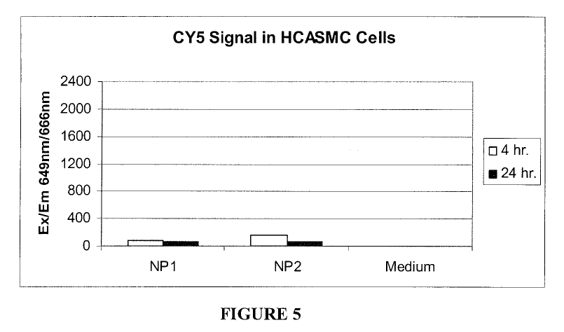

FIG. 5 shows that there was no uptake of PLGA nanoparticles by human cardiovascular arterial smooth muscle cells (HCASMC) again using CY5 as the reference standard. The HCASMC were cultured on 96 well cell culture plate followed by treating with various formulations of PLGA nanoparticles for 4 hours (white bar) or 24 hours (black bar). The HCASMC were washed in 1× PBS three times and then lysed in 50 μl cell lysis buffer for 15 minutes at room temperature. The CY5 signal of the HCASMC contents was evaluated at excitation/emission (Ex/Em) 649 nm/666 nm. The NP1 refers to nanoparticles having particle size 600 nm and NP2 refers to nanoparticles having particle size 1100 nm in FIG. 5, respectively.

### EXAMPLES

Certain embodiments of the present invention can be further illustrated by the following set forth examples, which are provided for illustrative purposes only and are not intended nor should they be construed as limiting the scope of this invention in any manner whatsoever.

### Example 1

### Preparation of Fluorescent Liposome (DSPG:DSPC:cholesterol:Ceremide 10; 1:3:1:1%):

A solution of DSPC (270 mg, 0.342 mmol) in chloroform (9 mL), DSPG (90 mg, 0.112 mmol) in chloroform (3 mL), cholesterol (45 mg, 0.116 mmol) in chloroform (1.5 mL) and ceramide 10 (2 mg, 1% total lipid) in chloroform (0.2 mL) was stirred in a round bottom flask, followed by addition of acid washed glass beads (6 g). The reaction mixture was stirred under vacuum at 60-90° C. to remove solvent. The flask was kept under vacuum overnight during which time a dry film formed. Fluorescent dextran (1.216 g) in deionized water (DI water) (4 mL), HEPES buffer (4 mL, 5 mM, pH 7.2), sodium chloride (152 mg) in DI water (1 mL) was added to the flask. The solution was stirred in a waterbath for 10 min. at 60° C. followed by hydration for 45 hrs. The mixture was purged with argon gas, liquid nitrogen for 5 min. followed by heating for 5 min. at 60° C. The process was repeated 5 times. The mixture was passed through a 400 nm filter 5 times followed by a 200 nm 5 times and finally a 100 nm filter 5 times using a Norther Lipids Inc. extruder at 60° C. The mixture was passed through a Sephadex-G100 column which was eluted with HEPS buffer (5 mM, NaCl 50 mM, total salt concentration about 55 mM).

### Example 2

### Preparation of PLGA Nanoparticles using the Modified Water-in-Oil-in-Water (W1/O/W2) Double Emulsion Technique:

A solution of PVA in DI water (150 mL) (water continuous phase) was prepared. A second solution of PVA in water (750 mL) was prepared and to this was added ApoA1 (77 mg) (W1). Then a solution of PLGA (180 mg) in methylene chloride (4 mL) (O) was added to the (W1) solution and the mixture was sonicated for one minute to form a W1/O emulsion. A solution of PVA in DI water (8 mL) (W2) was then added to the (W1/O) emulsion followed again by sonication to give a second emulsion (W1/O/W2), which was immediately poured into the continuous water phase, stirred at 500 rpm for 5 minutes and 300 rpm for 3 hours. The nanoparticles which formed were separated by ultra-centrifugation. The supernatant liquid was collected and discarded leaving precipitated nanoparticles as pellets. The nanoparticle pellets were washed twice with water and then suspended in water followed by lypohilization to provide the nanoparticles in powder form.

While the present invention has been described in terms of certain embodiments, other embodiments not expressly disclosed will, based in the disclosure herein, occur to those skilled in the art. Such embodiments are within the scope of this invention.

What is claimed:

1. An implantable medical device, comprising:
a device body having an exposed surface;
a drug reservoir layer disposed over at least a portion of the exposed surface of the device body;
a plurality of particles embedded in the drug reservoir layer;
one or more therapeutic agents encapsulated in the plurality of particles, wherein
the particles are surface-treated with a first substance capable of enhancing transport of the particles into a lipid-rich atherosclerotic lesion and a second substance capable of enhancing uptake of the particles into macrophages within the lesion.

2. The implantable medical device of claim 1, wherein the device is a stent.

3. The implantable medical device of claim 1, wherein the plurality of particles are selected from the group consisting of micelles, liposomes, polymerosomes, hydrogel particles and polymer particles.

4. The implantable medical device of claim 3, wherein the liposome has a particle size from about 80 nm to about 1 micron.

5. The implantable medical device of claim 1, wherein the first substance is selected from the group consisting of thiolated chitosan, TDMAC, PPAA, and combination thereof.

6. The implantable medical device of claim 1, wherein the second substance is selected from the group consisting of phospholipids, DSPG, PLA/PLGA, ceramide, and combination thereof.

7. A method of treating a vascular disease, comprising:
deploying in the vasculature of a patient in need thereof an implantable medical device, wherein the device comprises:
a device body having an exposed surface;
a drug reservoir layer disposed over at least a portion of the exposed surface of the device body;
a plurality of particles embedded in the drug reservoir layer;
one or more therapeutic agents encapsulated in the plurality of particles, wherein
the particles are surface-treated with a first substance capable of enhancing transport of the particles into a lipid-rich atherosclerotic lesion and a second substance capable of enhancing uptake of the particles into macrophages within the lesion.

8. The method of claim 7, wherein the device is a stent.

9. The method of claim 7, wherein the plurality of particles are selected from the group consisting of liposomes, micelles, polymerosomes, hydrogel particles and polymer particles.

10. The method of claim 9, wherein the liposome has a particle size from about 80 nm to about 1 micron.

11. The method of claim 7, wherein the first substance is selected from the group consisting of thiolated chitosan, TDMAC, PPAA, and combination thereof.

12. The method of claim 7, wherein the second substance is selected from the group consisting of phospholipids, DSPG, PLA/PLGA, ceramide, and combination thereof.

13. The method of claim 7, wherein the vascular disease is atherosclerosis.

14. The method of claim 7, wherein the vascular disease is restenosis.

15. The method of claim 7, wherein the vascular disease is vulnerable plaque.

16. The method of claim 7, wherein the vascular disease is peripheral vascular disease.

17. The method of claim 7, wherein the vascular disease is late stent thrombosis.
